(19) United States
(12) Patent Application Publication
(10) Pub. No.: US 2020/0022771 A1
(43) Pub. Date: Jan. 23, 2020

(54) STERILE PACKAGING FOR SURGICAL TOOLS

(71) Applicant: BioAccess Inc., Baltimore, MD (US)

(72) Inventor: Robert Chapolini, Baltimore, MD (US)

(21) Appl. No.: 16/337,876

(22) PCT Filed: Sep. 29, 2017
(86) PCT No.: PCT/US17/54591

§ 371 Date: Mar. 28, 2019

Related U.S. Application Data
(60) Provisional application No. 62/403,059, filed on Oct. 1, 2016.

(51) Int. Cl. A61B 50/30

(52) U.S. Cl. CPC A61B 50/30; A61B 2050/0058

(57) ABSTRACT

The present invention is package for a sterile tool, and a system for housing and using the tool in a sterile environment. The tool in its non-functioning state, lacking non-sterile components, is disposable. The system permits insertion of non-sterile elements of the tool into the tool while maintaining sterility of the outside portion of the tool. When the package is opened, a door on the tool automatically closes over the non-sterile element in the sleeve.

### I. CROSS-REFERENCE TO RELATED APPLICATIONS

This application claims the benefit under 35 U.S.C § 119(e) of U.S. Provisional Application No. 62/403,059 entitled “Sterile Packaging for Surgical Tools” filed 1 Oct. 2016, the entirety of each of which is incorporated by reference herein.

### II. FIELD OF THE INVENTION

This invention relates to a package or enclosure and/or system for packaging surgical tools, maintaining the tool in a sterile condition after a non-sterile/reusable component is inserted in the tool.

### III. BACKGROUND OF THE INVENTION

Medical and surgical devices continue to become more sophisticated and expensive. Typically, these instruments must be sterilized before use. Instruments that are reusable must be made very durable to withstand repeated sterilizing cycles and this adds considerable manufacturing costs. In order to reduce the cost of these instruments, disposable tools are desirable, but many disposable tools include non-reusable and/or non-sterilizable parts, e.g., a motor or a lithium battery.

Cordless surgical drills are frequently used in the operating room. These drills have replaceable battery packs which must be sterilized before use. Steam sterilization can substantially reduce the life expectancy of the battery pack. For example, a battery powered surgical drill typically has a removable, rechargeable battery pack. Although many of these battery packs can be steam sterilized, battery life can be extended by eliminating the sterilizing process and loading the battery into the drill aseptically.

Notwithstanding the usefulness of the above-described methods, a need still exists for packaging surgical tools that permits insertion of a non-sterile element into the sterilized tool without compromising the sterility of the operating theatre, the sterility of the tool, or the person handling the tool.

### IV. SUMMARY OF THE INVENTION

This invention provides an enclosure, method, and system for packaging a tool. The tool is sterile, configured to house a non-sterile/reusable component in the tool. Further, the sterility of the tool is maintained until it is removed from the package.

Optionally, the invention(s) may further include packaging the used tool and disposing all disposable parts of the tool.

The invention also includes an enclosure that is configured to house the sterile tool, and to insert the non-sterile element (e.g., a battery, a motor, or combinations thereof) into an interior sleeve of the tool. In some embodiments of the invention, the package or enclosure comprises one or more elements or features that permit access to the interior of the sleeve or an interior portion of the tool.

The present invention also includes using the packaging and the tool in combination to maintain tool sterility during and after inserting a non-sterile component into a portion of the tool. In some of these embodiments, the sterile tool may be removed from the packaging without compromising the sterility of the environment (e.g., a sterile field) and/or the sterility of the tool user or handler.

The present invention is also a method and system for placing a non-sterile element into a sleeve of a sterile medical device, whereby the non-sterile element does not contaminate the outside portion of the sterile tool. In some embodiments of the invention, the method comprises using a sterile tool having a non-sterile and/or disposable component.

The present invention may optionally include re-packaging the used (i.e., non-sterile) tool in the package, closing the package, and separating the non-disposable portion of the tool from the disposable or non-reusable portion of the tool. In these embodiments of the invention, the method may include removing the non-disposable portion of the tool, and disposing the package and the disposable portion of the tool.

The present invention also permits the assembly of a non-sterile component within a sterile component. The two parts, when assembled, form a complete medical device which is sterile on its outer surface. An example of a two component medical device is a disposable surgical drill. Typically, an apparatus such as a disposable drill is packaged as a sterile, single use, disposable medical device. A motor (or motor-battery assembly), typically re-usable and non-sterile, must be inserted into the handle of the drill to make it functional. The present invention permits insertion of the non-sterile/re-usable element into the sterile tool without any chance of contamination of the outside of the sterile tool. The invention can then be opened and the tool removed by a gloved operator. A hatch door on the sterile tool closes the handle portion and protects from any contact with the non-sterile element contained inside.

At the end of the surgical procedure, the hatch door is opened and the tool placed back in the package. The package is then closed, thereby containing all outer surfaces of the tool which may have come in contact with the patient. The reusable/non-disposable element(s) may then be removed and retained for future use. The package with the contaminated tool can then be discarded.

With the following description of the drawings, the apparatus should become evident to a person of ordinary skill in the art.

### V. BRIEF DESCRIPTION OF THE FIGURES

FIG. 1 is a top view of a closed package containing a medical tool, illustrating the door of the tool in a first packaged position or in a partially open position

FIG. 2 is a perspective view of a closed package, showing the inside of the sleeve of the tool, said inside portion being adapted to receive a non-disposable and/or non-sterile component of the tool.

FIG. 3 is a bottom view of the package and tool shown in FIG. 2, illustrating access through the package into an empty sleeve or portion of the tool.

FIG. 4 is a bottom side view of the closed package, showing the port in the package and the non-sterile portion of the tool inserted into the sleeve of the tool.

FIG. 5 is a side view of the package as the package is being opened, illustrating removing the tool, thereby freeing the door to automatically close over the opening in the sleeve of the tool.

FIG. 6 shows the tool completely removed from the package, with the hatch door automatically closed.

FIG. 7 shows the package containing the tool after use.

### VI. DETAILED DESCRIPTION OF THE INVENTION

The present invention is a method of inserting a non-sterile and/or re-usable component into a portion of a sterile tool while maintaining the sterility of the exterior of the tool. The method involves a package configured with access to an internal sleeve or portion of the tool. The method also involves an envelope or package configured so that opening the package automatically closes a cover or door on the internal portion of the tool, thereby enclosing the non-sterile element within the tool, and thereby isolating the element(s) from the sterile exterior of the tool.

In some embodiments of this method, the method includes isolating the user or handler of the tool from the non-sterile component.

The method may further include sealing the door or hatch, further isolating the non-sterile component from the user/handler, and thereby protecting the tool user/handler and patient from the non-sterile component.

In an optional embodiment of the method, the package may be configured so that when the used/non-sterile tool is returned to the package, the door is opened to a fully open position. In these embodiments, the method further includes removing the non-sterile and/or re-suable component from the tool through an aperture in the package. Some embodiments of the invention further include deposing of the package and/or used tool (i.e., without the non-sterile component therein).

In preferred embodiments of the method, the door is moveable between at least three positions: A) a first position, or a partially open position, or a first packaged position; B) closed; and C) fully open or a second packaged position. In most preferred embodiments of the invention, the closed position may also be further sealable.

The present invention also includes methods for packaging a device having a sterile component and a non-sterile component. An embodiment of the invention includes positioning the device in a package adapted to receive the device and adapted to permit access to a sleeve in the device, and inserting a non-sterilized component of the device into the sleeve using an access port in the package.

The present invention also includes methods for packaging a device having a disposable portion and a non-disposable portion. An embodiment of the invention includes positioning the device in a package adapted to receive the device and adapted to permit access to a sleeve in the device, and inserting a non-disposable component of the device into the sleeve using an access port in the package.

The present invention also includes methods for packaging a device having a sterile and disposable portion and a non-sterile non-disposable (and/or re-usable) component. An embodiment of the invention includes positioning the device in a package adapted to receive the device and adapted to permit access to a sleeve in the device, and inserting the non-sterilized non-disposable component of the device into the sleeve using an access port in the package.

An embodiment of the invention includes a method of using a sterile tool comprising a tool having a cover or hatch movable between multiple positions, and a package configured to enclose the tool with the door in at least one, a preferably two open positions. In these embodiments of the invention, the tool is positioned in a closed package with the door in a partially open position, wherein in this position, the door is under a spring tension. A non-sterile element such as a motor and/or battery can be placed into the tool through an aperture in the closed package. The package may then be opened, and when the tool is removed from the package, the door automatically closes over the sleeve containing the non-sterile element. A door knob or the like can be turned to close the door into a closed and sealable position. After completion of the tool's use, the tool may be returned to the tray, with the door or hatch moved to a fully open position, defined herein as the door being open and will not automatically close.

In another embodiment of the invention, the apparatus and method may also include re-packaging a used (e.g., non-sterile and/or contaminated with body fluid) device in the packaging, closing the packaging, and disposing of the package containing the used device. Typically, the disposal process will include disposing of the non-re-useable portions of the device. For example, in some embodiments of this aspect of the invention, a battery pack and/or motor may be removed from the device prior to re-packaging. In preferred embodiments of this aspect of the invention, a battery pack and/or motor may be removed from the device after re-packaging. In this way, at the end of the surgical procedure, all patient contact elements are disposed of. This combination of sterile and non-sterile components strikes a balance between the desirability of disposable medical instruments and devices with the reality of medical cost containment.

Examples of the method may include any one or more of the following features, alone or in any combination: providing a package, with or without the tool, that is encased in a bag or cover; removing the bag or cover from the packaging holding the tool; providing a sealable pouch or bag to enclose the packaging; providing an openable bag or pouch to cover the packaging; removing the bag or pouch from the packaging; providing the package with a removable element that covers a port in the package that allows access to the sleeve; providing the tool with a removable element that covers the internal portion of the tool; providing a system that includes a package with a removable element that covers a port in the package that allows access to the sleeve and providing the tool with a removable element that covers the internal portion of the tool; providing the package with a penetrable element or cover that covers a port in the package that allows access to the sleeve; providing the tool with a penetrable element or cover that covers the internal portion of the tool; providing a system that includes a package with a penetrable element that covers a port in the package that allows access to the sleeve and providing the tool with a penetrable element that covers the internal portion of the tool; as a step in a method, removing the removable element that covers the port in the packaging; as a step in a method, removing the removable element that covers the internal portion of the tool; as a step in a method, removing the removable element that covers the port in the packaging and removing the removable element that covers the internal portion of the tool; as a step in a method, penetrating the penetrable element that covers the port in the packaging; as a step in a method, penetrating the penetrable element that covers the internal portion of the tool; as a step in a method, penetrating the penetrable that covers the port in the packaging and penetrating the penetrable element that covers the internal portion of the tool; packaging the tool with the cover/hatch/door for the sleeve in a first open position (non-automatic closure); packaging the tool with the cover for the sleeve in a second open position (automatic closure); opening the packaging and thereby allowing the door to automatically cover the internal portion of the tool; opening the packaging and thereby allowing the handler to manually close the cover over the internal portion of the tool; opening the packaging, sliding the tool to a first position in the packaging that allows the door to close over the internal portion of the tool; opening the packaging, said packaging further comprising a sleeve or barrier protecting the handler from the internal portion of the tool, and sliding the tool to a first position in the packaging that allows the door to close over the internal portion of the tool; removing the tool comprising a closed cover over the internal portion from the packaging; sealing the closed cover over the internal portion; sealing the closed cover over the internal portion by screwing or bolting the cover to the tool; after opening the package, inserting one or more elements into the internal portion of the tool, covering and/or sealing the internal portion, then providing a fully functional or operable tool; providing a fully functional or operable tool have a component or components enclosed by a sterile portion of the tool; using the tool in a sterile environment; using the tool in a surgical or medical environment; returning the used tool to the package; closing the package over the used tool; removing one or more components from the used tool; removing one or more components from the used tool, through an aperture in the packaging; removing one or more non-sterilizable components from the used tool; removing one or more non-sterilizable components from the used tool, through an aperture in the packaging; removing one or more non-disposable components from the used tool; removing one or more non-disposable components from the used tool, through an aperture in the packaging; removing one or more non-sterilizable and non-disposable components from the used tool; removing one or more non-sterilizable and non-disposable components from the used tool, through an aperture in the packaging; disposing the packaging; disposing the packaging containing the disposable portion of the tool; disposing the packaging containing the tool; re-charging a battery or battery pack; reconditioning and reusing a motor or motor assembly; inserting a recharged battery or battery pack in a second tool; inserting a reusable motor or motor assembly in a second tool; providing a tool with a rechargeable battery or battery pack and a non-rechargeable battery of battery pack; and providing a tool with a disposable, non-rechargeable battery or battery pack.

The present invention is a package for a sterile tool, wherein the package is configured to enclose the tool, and the package includes a port allowing access to an inner portion of the tool.

The present invention is a housing, package, or enclosure for a surgical tool, wherein the outside of the tool is sterile, said tool comprising an inner portion or sleeve adapted and configured to receive and encase a non-sterile component. The non-sterile component is re-usable and/or not disposable. The package is configured to provide access to a sleeve in the tool, said sleeve encasing or housing the non-sterile component.

The component of the tool that is inserted into a portion of the tool may have one or more attributes, any or all of which make it desirable or beneficial that the component is not permitted to touch or contact the outside of the tool. An exemplary attribute includes non-sterility; a motor, for example, may be non-sterile or non-sterilizable. Another exemplary attribute may be re-usability; a rechargeable battery may be recharged and used in another tool. Another attribute might be both non-sterile and re-useable; a motor may be non-sterile and/or not sterilizable, and may be used in another tool. Further, a motor/battery assembly may be both non-sterile and reusable. It is intended that the invention should not be limited by the nature or number of these various attributes.

Some embodiments include providing a sterile tool packaged in a sterile enclosure or package. In some embodiments, the tool includes an internal portion and a door or hatch for covering the internal portion.

In one embodiment of the invention, the apparatus comprises a sterile enclosure, package, or envelope that houses a sterile device. In preferred embodiments of the invention, the package includes a port positioned to align or communicate with an internal portion of the tool. In the most preferred embodiments of the invention, the tool further includes a cap or hatch configured to close over the internal portion of the tool.

In preferred embodiments of the invention, one or both of the packaging and the tool are disposable. In the most preferred embodiments, a used tool enclosed by a package is disposable.

In one embodiment of the invention, a system comprises an enclosure suitable for enclosing or housing a sterile device or tool. In these embodiments, the enclosure also includes a port or access portion that permits access to the internal portion or sleeve of the tool when the tool is positioned in the housing. In the most preferred embodiments, the sterile tool further includes a cover, door, or hatch for closing or covering the internal portion of the tool or sleeve.

In some embodiments of the invention, the sterile device comprises a sleeve or the like having a cover in an open position when positioned in the housing, said sleeve having an inside portion adapted to communicate with a port or aperture in the sterile packaging. The port permits insertion of a non-sterile component, such as a battery, battery pack, and/or motor, into the sleeve of the sterile device.

In some embodiments, the package is configured to position or hold the door in its partially open position, also referred to herein as its first packaged position. In some embodiments, the package may be further configured to hold the door in a fully open position, also referred to herein as its second packaged position.

In some embodiments, the package may be further configured with a shield or the like that protects the user/handler as the tool is being removed from the package (i.e., during the period when the door is automatically closing over the internal sleeve.) In some embodiments, the package may be further configured with a shield or the like that protects the user/handler from being contaminated by the non-sterile component when the door to the tool is in a partially closed position or in a non-sealed condition.

In some embodiments, the package may be configured or adapted to include indents or spaces specifically configured to conform to the door of the sleeve. In some embodiments, the indents may closely conform to the open door. In preferred embodiments, the indents allow the door to move between a partially open position and a fully open position.

In the most preferred embodiments, the packaging and/or the packaging closed around a used or disposable tool, is disposable in its entirety. In these embodiments of the invention, the disposable or non-sterile portion of the tool is first removed from the package and/or tool.

An assembled device according to the invention is suitable for use in a sterile field. A sterile field refers to any location where contamination is undesirable and/or where contamination should or must be maintained at a minimum level. Exemplary environments include but are not limited to medical (e.g., surgery rooms) and electrical (electronics assembly rooms) environments. In a preferred embodiment of the invention, the sterile field is a surgical theater or operating room. Examples of the apparatus and system may include any one or more of the following features, alone or in combination: a cover over the packaging port; a penetrable cover over the packaging port; a package with a removable element that covers a port in the package that allows access to the sleeve; a tool with a removable element that covers the internal portion of the tool; a system that includes a package with a removable element that covers a port in the package that allows access to the sleeve and a tool with a removable element that covers the internal portion of the tool; a package with a penetrable element or cover that covers a port in the package that allows access to the sleeve; a tool with a penetrable element or cover that covers the internal portion of the tool; a system that includes a package with a penetrable element that covers a port in the package that allows access to the sleeve and a tool with a penetrable element that covers the internal portion of the tool; a tool with the cover/hatch/door for the sleeve in a first open position (non-automatic closure); a tool with the cover for the sleeve in a second open position (automatic closure); a door or cover configured to encase the inner portion of the tool, said door or cover closes automatically upon opening the package; a door or cover to close the inner portion of the tool, said cover being closable manually by the handler; packaging, that is configured with one or more elements or spaces that allow the tool to slide to a first position in the packaging; packaging that is configured with one or more elements or spaces that allow the door to close over the internal portion of the tool; packaging further comprising a sleeve or barrier protecting the handler from the internal portion of the tool, and sliding the tool to a first position in the packaging that allows the door to close over the internal portion of the tool; a fully functional or operable tool; a fully functional or operable tool have a component or components enclosed by a sterile portion of the tool; a tool suitable for use in a sterile environment; a tool suitable for use in a surgical or medical environment; a package configured for enclosing a used or non-sterile tool; a package configured for enclosing a used or non-sterile tool and configured with a port or access to an inner portion of the used tool; a package configured for enclosing a used or non-sterile tool; a package configured for enclosing a used or non-sterile tool and configured with a port or access to a reusable or non-disposable element(s) of the used tool; a package and portion of a tool that is disposable; a rechargeable battery or battery pack; a reusable or reconditionable a motor or motor assembly; a tool with a rechargeable battery or battery pack and a non-rechargeable battery of battery pack; and a tool with a disposable, non-rechargeable battery or battery pack; a sealable pouch or bag to enclose the packaging (with or without the tool); an openable pouch or bag to enclose the packaging, with or without the tool.

A device, apparatus, or system of the present invention may include one or more of the following, alone or in any combination: the enclosure port may be covered with a sterilizable cover; the packaging port may be covered by a removable cover (e.g., a peel away cover); the port may be covered by a removable and sterilizable cover the entire sterile package or a portion of the package that includes the port may be enclosed in an outer enclosure, such as a bag, pouch, or the like;

The present invention is also a package or tray configured to house a sterile tool, wherein the tool includes an openable and closeable door or hatch; wherein the package contains a form or pocket to house the tool with the door open in at least two positions. In most preferred embodiments of the invention, the tray includes a port allowing access to an inner portion of a sleeve in the tool, wherein said sleeve can be closed or covered by the door when the tool is removed from the package.

More particularly, the present invention comprises an outer enclosure that contains the sterile sleeve and holds it so that only the inside of the sterile sleeve can be accessed through an opening in the closed package. Inserting the non-sterile element into the package can be performed by an un-gloved (non-sterile) operator. The outside of the sterile sleeve cannot be inadvertently contaminated during the insertion procedure. After the non-sterile element has been inserted, the invention can be opened and the sterile sleeve, with the non-sterile medical device contained within, can be removed by an operator wearing sterile gloves.

The present invention is also a tool comprising an inner portion or sleeve enclosing an inner space, wherein the inner portion is configured to receive a non-sterile and/or reusable component of the tool. Exemplary components include but are not limited to a motor, one or more batteries; a motor/battery assembly; a battery pack; and combinations thereof. In these embodiments of the invention, the tool also comprises at least one door or cover configured to cover or close an inner portion of the tool. In preferred embodiments of the invention, the cover encases the component, thereby making the fully functional tool sterile.

The present invention is also an apparatus for packaging and sterilizing a device comprising an enclosure that maintains a device in a sterile condition and permits insertion of a non-sterile component of the device into a portion of the sterile device while maintaining the device's sterile condition.

The invention includes any sterile or sterilizable device that typically requires the use or insertion of a non-sterilized component. Exemplary sterile devices include but are not limited to drills, endoscopes, saws, suction devices, accessories to any of the above, and disposables for any of the above. Non-sterilized components include but are not limited to a battery, a battery pack, a motor, a motor/battery assembly, and the like.

An embodiment of the invention includes a tool having a cover or hatch movable between one or more open, closed, or sealed positions, and a package configured to enclose the tool with the door in one and preferably two open positions.

In these embodiments of the invention, possible door positions are: 1) fully open, where the door will not automatically close; 2) partially open, where the door is under spring tension and will close automatically, e.g., when removed from the tray; 3) closed, where the door closes or covers the sleeve, but does not seal it; and 4) fully closed, where the door is closed and sealed.

Some embodiments of the invention may include another position: after the tool has been returned to the enclosure/package after use, the cover to the inner portion open to any position that allows access to a removable element of the tool.

A package or packaging system of the invention may be adapted to receive a device. As used herein adapted to receive refers to forms and sub-structures of the package intended to contact the device and hold the device in position in the package. For example, the package may be molded to conform with the shape of the device. Alternatively, the package may include inserts of the like that have been molded or shaped to conform to the shape of the device. Typical inserts include one or more pieces of foam or the like shaped to conform to the shape of the device or a portion of the device. In some embodiments of the invention, adapted to receive the device includes one or more structures that engage a portion of the device, such as a sleeve. In these embodiments of the invention, these structures engage or form an integral portion of a port in the package.

In accordance with the present invention, the device includes one or more structures adapted to receive a non-sterile component of the functional device. As noted above, the non-sterile component typically refers to one of more batteries and/or a motor. As used herein, adapted to receive a non-sterile component refers to one or more structures in the device suitable for enclosing the non-sterile component and sealing the non-sterile component form the sterile component(s) until the sterility of the functional device is no longer a concern. Exemplary structures suitable for enclosing the non-sterile component include but are not limited to a sleeve, housing, conduit, hollow member, or any other suitable enclosure. In a preferred embodiment of the invention, the sterile device includes a hollow handle, the inside portion of which may be adapted to receive one or more batteries and/or a motor.

A device according to the present invention may also include structures that are configured for and/or are adapted to permit access to the device structure suitable for enclosing the non-sterile component. In a preferred embodiment of the invention, this structure is a port in the package. Typical structures for sealing or covering the port include but are not limited to a removable or penetrable seal, an outer package that covers the port, or an outer package that covers an inner package having a port.

The present invention may also include an alternative or another embodiment, where the package includes a second cover positioned over a portion of the sleeve of the tool. This second cover further protects and/or shields the capped end of the tool during the period when the tool is being removed from the package. This cover shields the capped end of the tool as the cap or door is automatically closing.

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” is used herein in the sense of “example,” rather than “ideal.” As used herein, the terms “about,” “substantially,” and “approximately,” indicate a range of values within +/−5% of the stated value unless otherwise stated.

Reference will now be made in detail to examples of the present disclosure described above and illustrated in the accompanying drawings. Wherever possible, the same reference numbers will be used throughout the drawings to refer to the same or like parts.

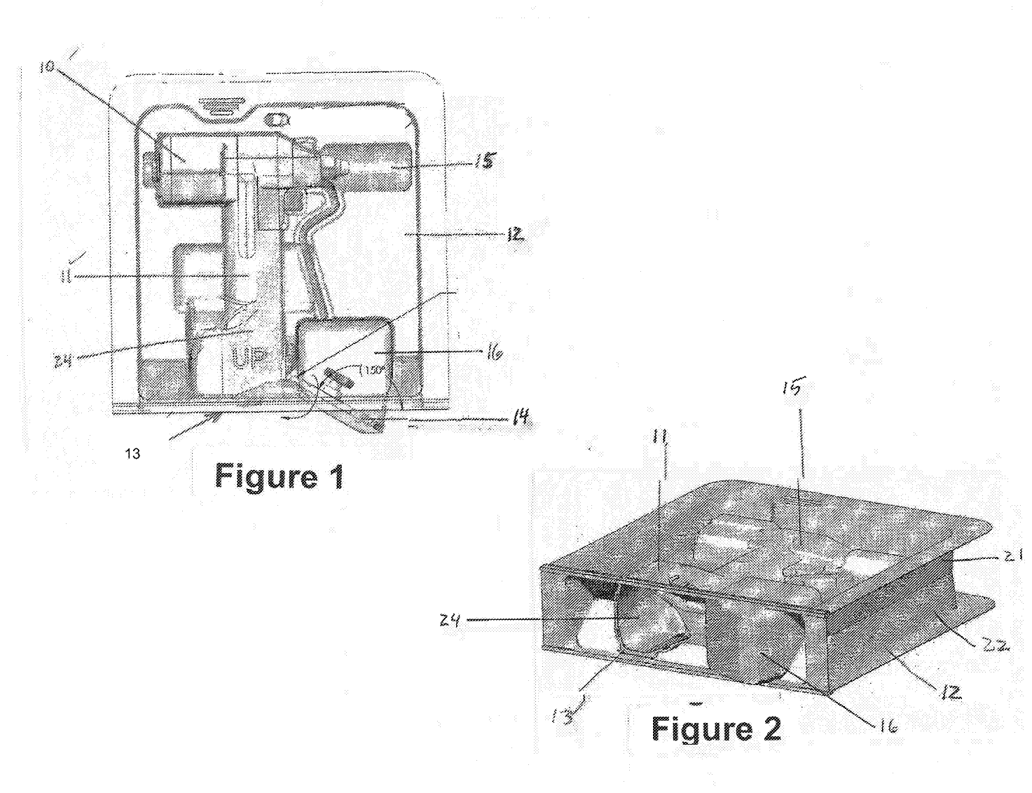

An exemplary packaging system and tool is shown in FIGS. 1 and 2. The package is generally denoted as 12, and is configured to receive, house, and position a sterile tool (such as drill 10) within the package. The package or housing includes an aperture or port 13 positioned adjacent a sleeve 11 of the tool. The port 13 allows access into the sleeve 11. In alternative embodiments, the port may be positioned in any location in the package, permitting access to any inner portion 24 of the tool.

A package or enclosure may be constructed of any material compatible with use in a sterile environment, and capable of withstanding sterilization. A wide variety of these containers are already known in the art. For example, these enclosures may be made from plasticized polyvinyl chloride, e.g. PVC plasticized with dioctylphthalate, diethylhexylphthalate, or trioctyltrimellitate; polyolefin, polyurethane, polyester, and/or polycarbonate. It is intended that the present invention is not limited by the type of material used to construct the container.

Enclosure 12 may be of any shape and size. One skilled in the art will recognize that the size and shape of device 10 will typically dictate the size and shape of enclosure 12 and any forms or configurations with the enclosure. Enclosure 12 may comprise one or more parts, sections, segments, or the like. In the embodiment of the invention shown in FIGS. 2 and 5, enclosure 12 may comprise a lower section 21 and an upper section 22 molded or joined by a hinge 23 region. As illustrated, section 21 and section 22 matingly engage when in a closed position.

Enclosure 12 may be variously configured to receive device 10. Preferably enclosure 12 is adapted by forming portions of the enclosure to engage and/or secure device 10 in place. One skilled in the art will recognize that a variety of structures, materials, and systems may be employed to position device 10 in enclosure 12. In a preferred embodiment of the invention, an interior section of enclosure 12 is molded to the shape of device 10. For example, FIG. 1 shows a void or cavity 15 adapted to receive device 10. FIGS. 1, 2, and 7 illustrate an enclosure having a cavity 16 configured to receive a cover 14 in a variety of its moveable, open positions.

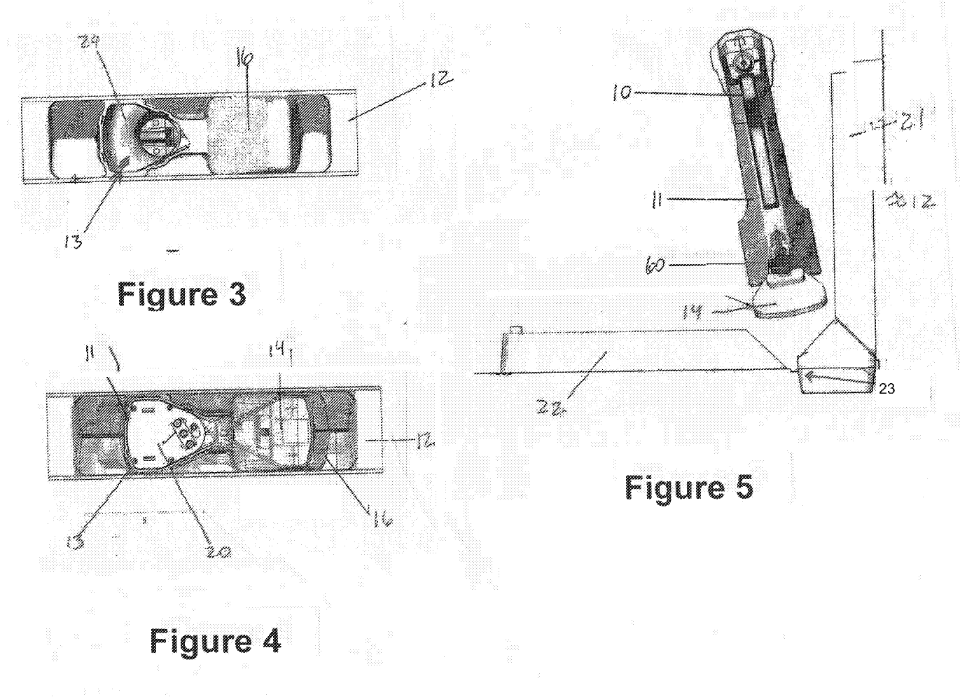

As shown in FIGS. 2 and 3, enclosure or packaging 12 is configured with a port or opening 13 that is shaped to allow access to an interior portion 24 of the tool 10. In preferred embodiments, opening 13 is positioned adjacent to, contiguous with, or proximate an inside portion 24 of sleeve 11 of tool 10.

Packaging 12, in the most preferred embodiments, may optionally include a removable cover (not illustrated) configured to cover all or a portion of the bottom of the packaging, or to temporarily cover the inner portion of the tool, or combinations thereof. In optional embodiments, packaging 12 may be contained within a sealed pouch, alone or in combination with a cover over the port or a cover over the inner portion.

FIG. 4 shows packaging 12 enclosing tool 10 with a reusable, non-sterilized and/or non-disposable element 20 positioned within the inner portion 24 of tool 10. In the illustrated embodiment, element 20 is a motor.

As noted above, enclosure 12 may comprise an integral structure, two or more mated and/or matable sections, or combinations thereof. A preferred embodiment of the invention comprises enclosure 12 comprising lower 21 and upper 22 sections (see for example, FIGS. 2 and 5). FIG. 5 shows enclosure 12 in its open position. A releasable capture mechanism, not shown, keeps the lower section 21 and the upper section 22 matingly engaged until the enclosed device 10 is needed. In the illustrated embodiment, enclosure 12 is openable, preferably using a hinge 23 or the like to provide access to the device 10 contained within.

In the embodiments of the invention shown in FIGS. 1, 2, and 4, sleeve 11 of device 10 is positioned in alignment with, adjacent to, and/or engaged with port 13. In a most preferred embodiment of the invention, port 13 permits access to an inner portion of sleeve 11 while enclosure 12 is closed. In the illustrated embodiments, sleeve 11 is a hollow handle of device 10. As shown, sleeve 11 of device 10 is positioned in the sterile enclosure 12 so that an open end of the handle is aligned with port 13 in the side of the enclosure 12. One skilled in the art will readily recognize that a variety of structures may be used to properly position the open end of sleeve 11 in relation to port 13.

The present invention also includes a method for combining a non-sterile component of a device with a sterile component of the device while maintaining the sterility of the sterile component. Exemplary structures that illustrate the methods of the invention are shown in FIGS. 3-7.

As is well known to practitioners in the art, the protocols for maintaining the sterility of a device 10 until it is operable and/or ready for use are varied. Typically, these protocols involve a single technician or handler performing certain actions while not sterile (in medical parlance, un-gloved) and other actions while sterile (e.g., gloved), or the coordinated actions between a sterile technician and a non-sterile technician. The essence of these actions is that a non-sterile technician should not touch a sterile device or enter a sterile field, and likewise, a sterile technician should not touch a non-sterile device. The various embodiments of the present invention accommodate all of these protocols.

An exemplary use or function of the system of the present invention includes providing a package, as described above, and a sterile but not-operable tool, as described above. If the sleeve or packaging includes a protective cover, this covering is first removed or penetrated, permitting access to the inner portion of the tool.

A non-disposable, non-sterile, and/or reusable element (e.g., motor, battery or motor-battery assembly) may then be placed into the sleeve of the tool, through the aperture in the closed package. In preferred embodiments of the invention, the motor engages mechanically or electrically with device 10 or the power source. In the most preferred embodiments, the connection between the non-sterile motor and the sterile portion of the tool is through an aseptic connection, thereby maintaining a sterile barrier. The package may then be opened.

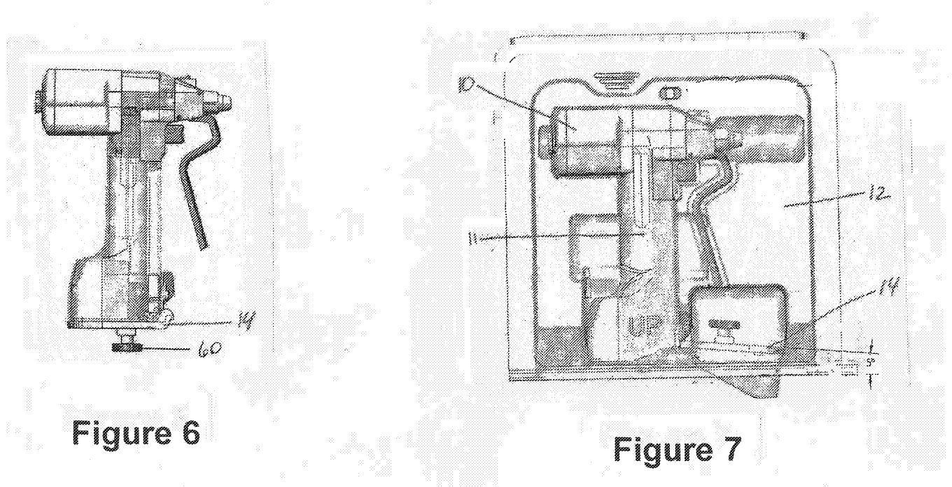

As shown in FIGS. 5 and 6, when the tool is removed from the package, the cover 14 for the sleeve automatically closes, covering the sleeve in a closed but not sealed position. The door may then by locked into a sealed position, after which, as defined herein, the tool is operable and ready for use in a sterile environment. In the illustration shown in FIG. 6, the cover 14 is sealed by tightening a lock, screw, or tightening mechanism 60.

After use, the tool will be non-sterile and may be placed back into the package, as shown in FIG. 7. Prior to placing the tool in the package, the door covering the sleeve may be unlocked and opened, and the motor or motor assembly removed from the sleeve. In some embodiments of the invention, the door may be moved to a fully open position, and the tool placed into the package. The package and tool may then be disposed of.

After use, the non-sterile component can be removed from the device 10 through aperture 13 of packaging 12, and reused one or more times.

In a further embodiment of the invention, once the operator has finished using device 10, it may be re-packaged in enclosure 12 to insure that others are protected from the now-contaminated device. In this embodiment of the invention, the intent is to retain the contamination inside the enclosure, close the enclosure, remove any reusable or non-disposable components, and transport the closed enclosure containing the contaminated device to a treatment center. Typically, any re-useable components of the device, or components that should not be sterilized, are separated from the device. The remainder of the device, e.g., the sleeve, handle, and/or housing, may be disposed or may be re-sterilized prior to use again.

In accordance with the present invention, the package as noted above is configured to position and hold the tool in the package. In the embodiment shown in FIG. 1, the tool is immobilized within the package. In an alternative embodiment, the package contains spaces or the like so that the tool may have freedom of movement away from the port or aperture. In this embodiment of the invention, the tool may be moved away from the aperture while the package is still closed; the movement away from the aperture allows the door or hatch to automatically close over the sleeve. This embodiment may also optionally include a tab or pull connected to the tool for pulling the tool away from the aperture.

In these embodiments of the invention, the sleeve of the tool is capped without opening the package. Once the cap is in place, the package may be opened, the tool removed, and the cap locked/sealed in place.

One skilled in the art will recognize that the invention as described here may be reconfigured into different combinations, elements, and processes which are included within the scope of the invention.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the disclosure is not to be considered as limited by the foregoing description.

We claim:

1. A method of using a medical device in a sterile environment, comprising:
A. providing a medical tool having a sleeve and a door for covering an inner portion of the sleeve;
B. providing a package adapted to conform to the shape of the tool, said package having a port that provides communication to said inner portion; said package configured to keep the door in a partially open position;
C. inserting a non-sterile element through said port into the inner portion;
D. opening the package, thereby automatically releasing the door and allowing the door to cover the inner portion of the sleeve.

2. The method of claim 1 wherein step A comprises providing a sterile medical tool that is not fully operable.

3. The method of claim 1 wherein step C comprises inserting a battery, a motor, or a battery-motor assembly.

4. The method of claim 3 wherein after step C, the tool is operable.

5. The method of claim 1 further comprising Step E comprising sealing the door over the sleeve.

6. The method of claim 5 wherein after Step E, the tool is operable in a sterile environment.

7. A packaging system for housing and using a tool in a sterile environment, comprising:
A. a non-operable and sterile tool comprising a hollow sleeve and a door for covering the sleeve;
B. a package for enclosing said tool, wherein the package comprises an aperture configured to allow access to the sleeve when the package is closed; and
wherein the package is configured to maintain the door in an open and automatically closeable position.

8. The packaging system of claim 7 further comprising a package configured to maintain the door in a fully open and not automatically closeable position.

9. The packaging system of claim 7 further comprising space for the tool to be moved away from the aperture, whereby movement of the tool allows the door to close over the sleeve without opening the package.

10. The packaging system of claim 9 further comprising a pull connected to the tool for pulling the tool away from the aperture.

11. A method for packaging a medical tool comprising:
A. providing a tool comprising an inner portion and a cover for said inner portion;
B. providing packaging configured to enclose said tool, said packaging comprising a port with access to said inner portion;
C. inserting an element into said inner portion; and
D. opening said packaging, thereby allowing said cover to cover said inner portion.

12. The method of claim 11 wherein inserting an element into said inner portion produces a fully functional tool.

13. The method of claim 11 wherein the element is selected from one or more of the following: a disposable element; a non-sterile element; a reusable element; a rechargeable element; and a non-rechargeable element.

14. The method of claim 11 further comprising, in step B, providing a package having a covered port.

15. The method of claim 1 further comprising, in step B, providing a package having a covered port.

16. The system of claim 7 further comprising a cover over said sleeve, said cover being removable or penetrable to gain access to an inner portion of said sleeve.
